(19) United States
(12) Patent Application Publication
(10) Pub. No.: US 2012/0045399 A1
(43) Pub. Date: Feb. 23, 2012

(54) POSITIVELY-CHARGED SUPERPARAMAGNETIC IRON OXIDE NANOPARTICLE, CONTRAST AGENT USING THE SAME AND METHOD OF PREPARING THE SAME

(75) Inventors: Yongdoo CHOI, Gyeonggi-do (KR); Hyunjin Kim, Gyeonggi-do (KR); Yun-Hee Kim, Gyeonggi-do (KR); Daehong Kim, Gyeonggi-do (KR); Inhoo Kim, Gyeonggi-do (KR)

(73) Assignee: National Cancer Center

(21) Appl. No.: 13/013,193

(22) Filed: Jan. 25, 2011

(30) Foreign Application Priority Data
Aug. 19, 2010 (KR) 2010-0080216

(51) Int. Cl. A61K 49/18; B05D 5/12; C12Q 1/02; A61K 49/00; C09K 11/00; B82Y 15/00; B82Y 5/00

(52) U.S. Cl. 424/9.323; 424/9.6; 427/127; 428/407; 435/29; 977/773; 977/930; 977/927

(57) ABSTRACT

Provided are a positively-charged superparamagnetic iron oxide nanoparticle (SPION), a contrast agent using the same, and a method of preparing the same. The positively-charged SPION includes a SPION, a polymer layer including a polymer containing many carboxyl groups coated on a surface of the SPION, and a cationic material coupled via an amide bond to a surface of the polymer layer. Therefore, the SPION may be prepared in a simple and reproducible process to have hydrophilicity and a strong positive charge. The prepared positively-charged SPION may have high uptake into a cell and stability, and be used in various applications as an effective contrast agent through non-invasive in vivo imaging.

### CROSS-REFERENCE TO RELATED APPLICATION

This application claims priority to and the benefit of Korean Patent Application No. 2010-0080216, filed on Aug. 19, 2010, the disclosure of which is incorporated herein by reference in its entirety.

### BACKGROUND

1. Field of the Invention

The present invention relates to a paramagnetic nanoparticle, a use thereof and a method of preparing the same, and more particularly, to a superparamagnetic iron oxide nanoparticle (SPION) whose surface is modified with a positive charge, a contrast agent using the same, and a method of preparing the same.

2. Discussion of Related Art

There are ongoing attempts to use cells as therapeutic agents for diseases including autoimmune diseases, neurodegenerative disorders and cancer. For example, human mesenchymal stem cells (hMSCs) show great potential for tissue regeneration [Bussolati B, J Nephrol 2006;19:706-709]. In cell therapies, it is very important to determine if cells administered to a patient are sufficiently delivered to a target position and obtain information on the number of the cells delivered to the target position. One of the most frequently used method to achieve such an object includes labeling stem cells with superparamagnetic iron oxide nanoparticles (SPIONs), which are a magnetic resonance imaging (MRI) contrast agent, administering the labeled stem cells to an individual, and non-invasively in vivo tracking the movement of the cells by MRI. To label stem cells with superparamagnetic nanoparticles, which are an MRI contrast agent, various techniques including lipofection, endocytosis, electroporation, and magnetofection may be used. Usually, cells are labeled with a mixture of SPIONs whose surface is coated with negatively-charged dextran and cationic polypeptides such as protamine sulfate or polylysine in a predetermined ratio for 24 to 36 hours [Arbab A S, et al. Blood, 2004. 15;104(4):1217-23]. It has been known that such a method is accompanied with a tedious task of finding the optimum conditions because cytotoxicity and labeling efficiency are dependent on the concentration and mixing ratio of the cationic polypeptides and the SPIONs, and has a negative effect on cells because of long-term cell treatment. As reported in various literatures, a cationic material serves to stimulate intracellular delivery. Accordingly, when the surface of superparamagnetic nanoparticles already has a sufficiently strong positive charge, the cells may be simply and efficiently labeled with the superparamagnetic nanoparticles without the need for an adjuvant such as protamine sulfate.

### SUMMARY OF THE INVENTION

The present invention is directed to a positively-charged SPION and a contrast agent using the same.

The present invention is also directed to a method of preparing a positively-charged SPION.

In one aspect, a positively-charged SPION is provided. The positively-charged SPION includes a SPION, a polymer layer including a polymer containing many carboxyl groups coated on a surface of the SPION, and a cationic material coupled via an amide bond to the surface of the polymer layer.

The positively-charged SPION may have a surface zeta potential of +30 mV or more, and an average diameter of 10 to 500 nm.

The polymer included in the polymer layer may be selected from polyacrylic acid, polymethacrylic acid, polyitaconic acid, and derivatives thereof.

The cationic material binding to the surface of the polymer layer may be quaternary ammonium containing an amine group.

The SPION may include maghemite (γ-Fe₂O₃) or magnetite (Fe₃O₄), and further include at least one selected from manganese (Mn), cobalt (Co), nickel (Ni), zinc (Zn) and gadolinium (Gd).

In addition, the positively-charged SPION may further include a fluorescent dye coupled via an amide bond to the surface of the polymer layer, and the fluorescent dye may be selected from rhodamine, bodipy, Alexa Fluor, cyanine and derivatives thereof.

In another aspect, a contrast agent including the above-mentioned positively-charged SPION is provided.

The contrast agent may be used for MRI, optical imaging, or MRI and optical imaging.

In still another aspect, a method of preparing a positively-charged SPION is provided. The method includes (a) preparing a SPION whose surface is coated with a hydrophobic ligand; (b) forming a hydrophilic polymer layer by substituting the hydrophobic ligand coated on the surface of the SPION with a polymer containing many carboxyl groups; and (c) forming an amide bond by reaction of the carboxyl groups exposed on the surface of the hydrophilic polymer layer with the cationic material containing an amine group.

Operation (c) may further include forming an amide bond by reaction of the carboxyl groups exposed on the surface of the hydrophilic polymer layer with a fluorescent dye.

### BRIEF DESCRIPTION OF THE DRAWINGS

The above and other objects, features and advantages of the present invention will become more apparent to those of ordinary skill in the art by describing in detail exemplary embodiments thereof with reference to the adhered drawings, in which:

FIG. 1 is a schematic view exemplifying a structure of a positively-charged SPION according to an exemplary embodiment of the present invention;

FIGS. 2A and 2B are transmission electron microscopic (TEM) images of 2-aminoethyl-trimethyl ammonium (TMA)-SPIONs;

FIG. 3 is a graph showing sizes of TMA-SPIONs and Feridex dispersed in deionized distilled water, as measured using a particle size analyzer;

FIG. 4 are images taken 22 hours after TMA-SPIONs are added to a phosphate buffer solution (PBS) and a PBS containing a fetus bovine serum (FBS);

FIG. 5 is a graph of comparison of relaxivity between TMA-SPIONs and Feridex;

FIG. 6 is T2-weighted MR images of distilled water, and Feridex and TMA-SPION aqueous solutions;

FIGS. 7A to 7C are images of human mesenchymal stem cells (hMSCs) taken after the hMSCs are treated with a cell culture (control), Feridex and TMA-SPION and stained with Prussian blue;

FIG. 8 is a graph of cell viability of stem cells according to concentrations of treated TMA-SPIONs and Feridex;

FIGS. 9A and 9B are white-light images and MR images of hMSCs labeled with SPIONs;

FIG. 10 is a graph obtained by comparing fluorescence intensity between TMA-SPIONs coupled with a fluorescent dye and fluorescent dye-free TMA-SPIONs; and

FIGS. 11A to 11D are MR images taken hourly to show whether TMA-SPION-labeled stem cells are accumulated in a cerebral infarction region in a mouse cerebral infarction model using Rose Bengal (Dark areas in the images indicated by arrows in FIGS. 11B to 11D are regions having TMA-SPION labeled stem cells).

### DETAILED DESCRIPTION OF EXEMPLARY EMBODIMENTS

Hereinafter, the present invention will be described with reference to examples and comparative examples in detail. However, the present invention is not limited to these examples. The exemplary embodiments described herein are provided to sufficiently deliver the spirit of the present invention to those of ordinary skill in the art. In the accompanying drawings, thicknesses of layers and regions may be exaggerated, reduced or omitted for the convenience of the description. Throughout the specifications, the same reference numerals indicate the same elements. When it is determined that the detailed descriptions for the known functions or configurations related to the present invention may depart from the substance of the present invention, the detailed descriptions will be omitted.

According to an exemplary embodiment of the present invention, a positively-charged SPION is provided.

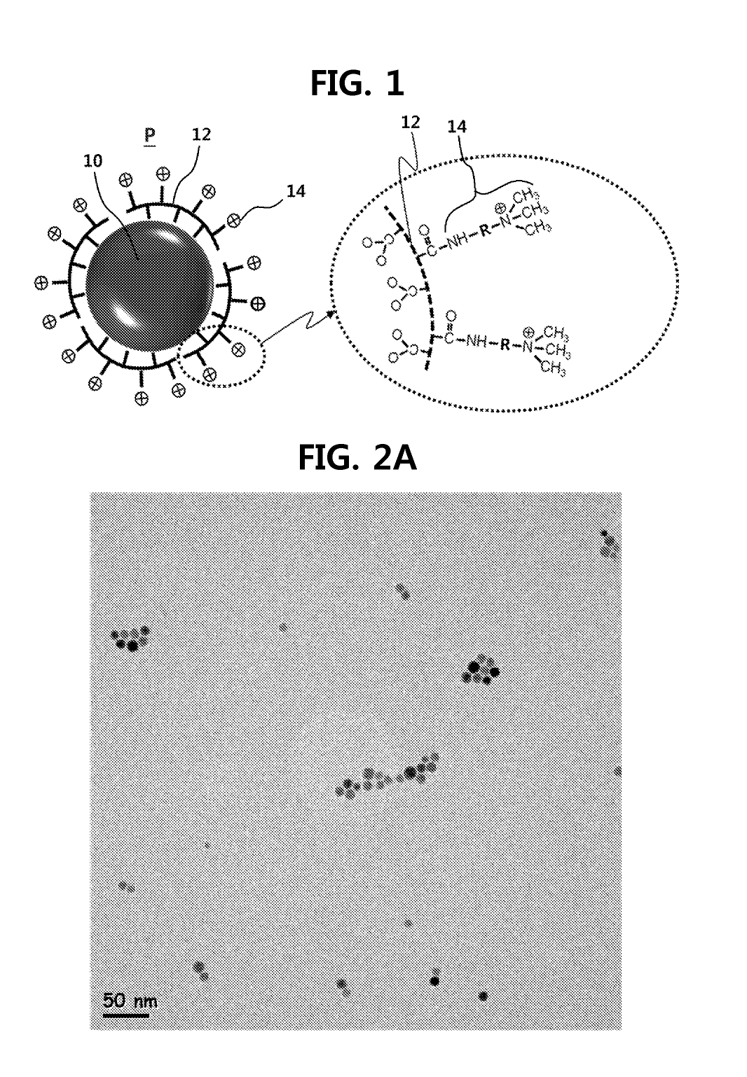

FIG. 1 is a schematic view exemplifying a structure of a positively-charged SPION according to this exemplary embodiment.

Referring to FIG. 1, the positively-charged SPION (P) includes a SPION 10 located in a central region (core) thereof, a polymer layer 12 including a polymer containing many carboxyl groups, which is coated on a surface of the SPION 10, and a cationic material 14 coupled via an amide bond to a surface of the polymer layer 12.

The SPION 10 located in the core includes maghemite (γ-Fe₂O₃) or magnetite (Fe₃O₄). Such a SPION 10 is magnetized when an external magnetic field is applied, and remnant magnetism disappears when the external magnetic field is removed. Therefore, the SPION 10 has no negative effects due to the remnant magnetism and excellent biocompatibility due to in vivo biodegradability, such that it can be used as a cell labeling material for MRI tracking of a therapeutic cell. In addition, the SPION 10 may further include at least one selected from manganese (Mn), cobalt (Co), nickel (Ni), zinc (Zn) and gadolinium (Gd), when necessary, to obtain hyperenhancement.

The polymer constituting the polymer layer 12 coated on the surface of the SPION 10 contains many carboxyl groups. Since the carboxyl groups form a coordinate bond with the SPION 10 at a multiple binding point, coating stability of the polymer layer 12 may be increased, and the SPION 10 may be uniformly dispersed in an aqueous solution by endowing the SPION 10 with hydrophilicity. The polymer containing many carboxyl groups may be polyacrylic acid, polymethacrylic acid, polyitaconic acid, and derivatives thereof. However, the present invention is not limited to these examples, and thus various biocompatible polymers containing carboxyl groups may be used.

The cationic material 14 may stably bind via an amide bond to the surface of the polymer layer 12, resulting in the SPION having a strong positive charge. The cationic material may be, for example, quaternary ammonium containing an amine group, which may form an amide bond by reaction with a carboxyl group that does not form a coordinate bond with the SPION 10 among the carboxyl groups included in the polymer layer 12. In FIG. 1, an example of the quaternary ammonium containing the amine group is a material in which a methyl group is bound to a nitrogen atom having a positive charge. However, the present invention is not limited thereto, and in addition to the methyl group, longer hydrocarbon group or an oxygen-containing hydrocarbon group (for example, polyoxyethylene) may be used, without departing from a range where a material does not lose its solubility. In FIG. 1, R indicates a hydrocarbon chain.

The surface of the positively-charged SPION (P) may have a zeta potential of +30 mV or more, and maintain very stable dispersibility in an aqueous solution due to strong repulsive power between nanoparticles. The positively-charged SPION (P) may have an average diameter of 10 to 500 nm, and a suitable size depending on the use of a contrast agent used within such a diameter range.

In addition, the positively-charged SPION (P) may further include a fluorescent dye binding to the surface of the polymer layer 12 (not shown). As the fluorescent dye is introduced, the positively-charged SPION (P) may be used as an optical imaging contrast agent as well as an MRI contract agent. The fluorescent dye may be coupled via an amide bond to the surface of the polymer layer 12 to form a strong bond with the SPION, and thereby may remain on the surface of the SPION after in vivo injection. The fluorescent dye may be rhodamine, bodipy, Alexa Fluor, cyanine, and derivatives thereof, but the present invention is not limited thereto.

According to another exemplary embodiment of the present invention, a method of preparing a positively-charged SPION is provided. The method includes (a) preparing a SPION whose surface is coated with a hydrophobic ligand; (b) forming a hydrophilic polymer layer by substituting the hydrophobic ligand coated on the surface of the SPION with a polymer containing many carboxyl groups; and (c) forming an amide bond by reaction of the carboxyl groups exposed on the surface of the hydrophilic polymer layer with a cationic material containing an amine group.

Operation (a) may be performed by various known methods including co-precipitation, thermal decomposition, hydrothermal synthesis and microemulsion, and preferably, thermal decomposition. By the thermal decomposition, the size of the SPION can be precisely controlled, the size differentiation of the SPION is uniform, and the crystallinity of the particles is high, resulting in an increase in a magnetic property. Here, since a surface of the SPION synthesized on an organic solvent is coated with a hydrophobic ligand, for example, a fatty acid such as oleic acid or lauric acid, a process of modifying the surface of the SPION into a hydrophilic surface is necessary for the SPION to be used as a contrast agent available in vivo.

Operation (b) may be performed by ligand substitution. Specifically, the SPIONs coated with the hydrophobic ligand prepared in operation (a) may be mixed with a hydrophilic polymer containing many carboxyl groups in a polar organic solvent and then heated, thereby substituting the hydrophobic ligand with the hydrophilic polymer. The polar organic solvent may be a glycol-based organic solvent such as ethylene glycol, diethyleneglycol or triethyleneglycol, and the hydrophilic polymer may be polyacrylic acid, polymethacrylic acid, polyitaconic acid or a derivative thereof. However, the present invention is not limited thereto.

Operation (c) is a process of modifying the SPION having a surface characteristic of a negative charge due to the carboxyl groups. A cationic material introduced by this operation may be coupled via an amide bond to a polymer layer coated on the SPION, thereby maintaining a stable bond. The used cationic material may be quaternary ammonium.

Meanwhile, operation (c) may further include a reaction binding a fluorescent dye to the surface of the hydrophilic polymer layer, in addition to the reaction of binding the cationic material to the surface of the hydrophilic polymer layer. In this case, the fluorescent dye is preferably coupled via an amide bond with a carboxyl group exposed on the surface of the hydrophilic polymer layer. The fluorescent dye may be, but not limited to rhodamine, bodipy, Alexa Fluor, cyanine, or a derivative thereof.

Hereinafter, to help with understanding of the present invention, exemplary examples are suggested. The following experimental examples are merely provided to help with understanding of the present invention, not to limit the present invention.

### Preparation Example 1

### Preparation of SPIONs Whose Surface is Modified With Positive Charge

Preparation of SPIONs Coated With Hydrophilic Polymer

The oleic acid-coated superparamagnetic iron oxide (Fe₃O₄) nanoparticle (OA-SPION) was synthesized according to the procedure of Park et al [Nature materials, 3:891-895 (2004)]. A surface of OA-SPION had a hydrophobic surface characteristic. Subsequently, the oleic acid was substituted with a hydrophilic polymer, polyacrylic acid (PAA), by the following method.

First, 200 mg of oleic acid-coated SPIONs (OA-SPION) was added to 2 ml of toluene and stirred for one day at 25° C. to be well dispersed. 16 ml of diethylene glycol (DEG) and 2 g of PAA (Mw 1,800, Sigma-Aldrich) were added to a round-bottom flask with a neck, stirred for 5 minutes under an argon (Ar) gas flow, and heated at 110° C. for 30 minutes to completely dissolve PAA in DEG. 2 ml of the toluene solution in which the OA-SPIONs were dispersed was injected into the flask using a syringe, and the reaction temperature was increased to 240° C. for 6-hour reaction. During the reaction, Ar gas was continuously provided to the flask. After the reaction, the temperature of the solution was slowly cooled at room temperature to lower the temperature. A strong acid solution was prepared by adding 2 ml of hydrochloric acid (HCl) to 150 ml of deionized distilled water to adjust a pH to approximately 2.6, and the reaction product obtained above was added to the strong acid solution to induce precipitation of PAA-coated SPIONs (PAA-SPIONs). The precipitated SPIONs were centrifuged for 15 minutes at 7000 rpm, a supernatant was discarded, and then 20 ml of 100 mM NaOH was added thereto to redisperse the precipitate. The nanoparticle dispersion was poured over a dialysis membrane (Spectrumlabs, Inc., MWCO 50,000 Daltons), and dialyzed in deionized distilled water for 18 hours to remove PAA, which was not bound to the surface of the SPION. After the dialysis, nanoparticles having a size larger than 200 nm were removed using a syringe filter (Pall Corporation, GHP acrodisc, 0.2 μm), and the aqueous solution was freeze-dried using liquid nitrogen. When the reaction was performed with 200 mg of OA-SPIONs, an average of 100 mg of PAA-SPIONs having a surface negative charge were obtained (a yield of approximately 50%).

Preparation of SPIONs Whose Surface was Modified With Positive Charge

100 mg of PAA-SPIONs having a nanoparticle surface substituted with polyacrylic acid were dispersed in 10 ml of deionized distilled water, 200 mg of 2-aminoethyl trimethyl ammonium (TMA) was added to the aqueous solution in which the PAA-SPIONs were dispersed, and then stirred at room temperature for 30 minutes. Afterwards, 0.2 M 1-Ethyl-3-(3-dimethylaminopropyl)-carbodiimide (EDC) was added to 500 μl of 0.1 M MES buffer (pH 4.7), and the resulting solution was added to the aqueous solution in which the PAA-SPIONs were dispersed and further stirred for 2 hours. The resulting solution was added over a dialysis membrane (Spectrumlabs, Inc., MWCO 50,000 daltons), and dialyzed in deionized distilled water for 18 hours to remove TMAs that were not bound to the surface of the SPION. The aqueous solution in which SPIONs whose surface was substituted with cationic TMA, TMA-SPIONs, were dispersed was freeze-dried using liquid nitrogen into a powder, and stored at 4° C. When the reaction was performed with 100 mg of PAA-SPIONs, an average of 50 mg of TMA-SPIONs in which a surface charge was modified with a positive charge was obtained (a yield of approximately 50%).

### Analysis Example 1

### Analyses of Shapes, Potentials, and Stabilites of PAA-SPIONs and TMA-SPIONs

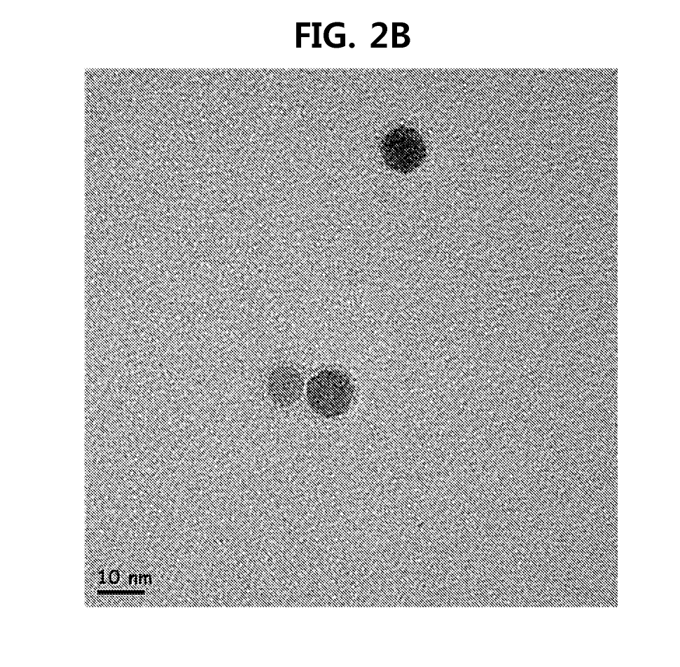

An aqueous solution in which PAA-SPIONs and TMA-SPIONs were dispersed in deionized distilled water was dispensed in a 400-mesh copper grid (Ultrathin carbon film, product No. 01824, TED PELLA, Inc.), dried for one day, and then observed with a TEM (300 kV, FEI Tecnai: F3OST). The SPION was formed in a circular shape, which was not changed before and after the substitution with PAA or TMA. FIGS. 2A and 2B are TEM images of TMA-SPIONs. It was seen that the core of the SPION has a size of approximately 9.6 nm, and a uniform circular shape.

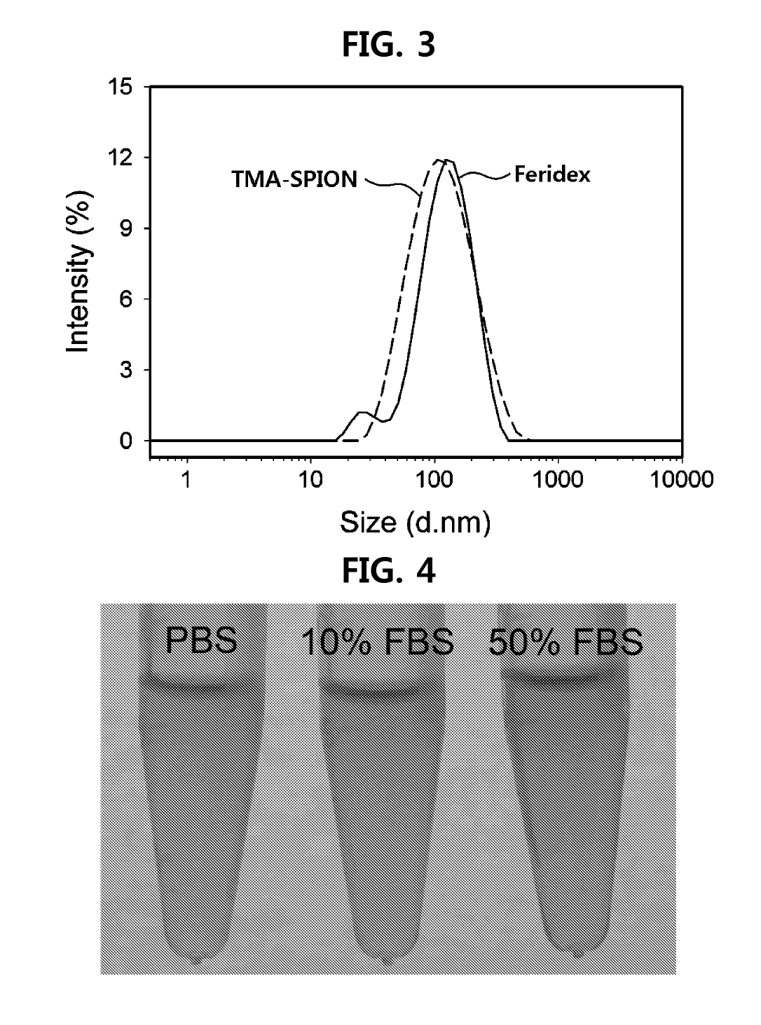

After a nanoparticle powder was diluted in deionized distilled water, the hydrodynamic size and surface zeta potential of each SPION were analyzed using a particle size analyzer (Nano Zetasizer; Malvern Instruments, Malvern, UK). The average size of the TMA-SPION was approximately 101 nm, which was similar to Feridex (Advanced Magnetics, Inc.) in aspects to size distribution (see FIG. 3). It was seen that the zeta potential of PAA-SPION was −41.6±5.3 mV, and the surface of the SPION had a strong negative charge. It was also seen that the zeta potential of the TMA-SPION was +40.0±2.2 mV, and the surface of the SPION was changed into a positive charge from a negative charge. It has been seen that the SPION having a surface zeta potential of +30 mV or more were very stably dispersed in an aqueous solution, and the TMA-SPIONs having a zeta potential of approximately +40 mV maintained stable dispersion in distilled water without a change in size for 200 days.

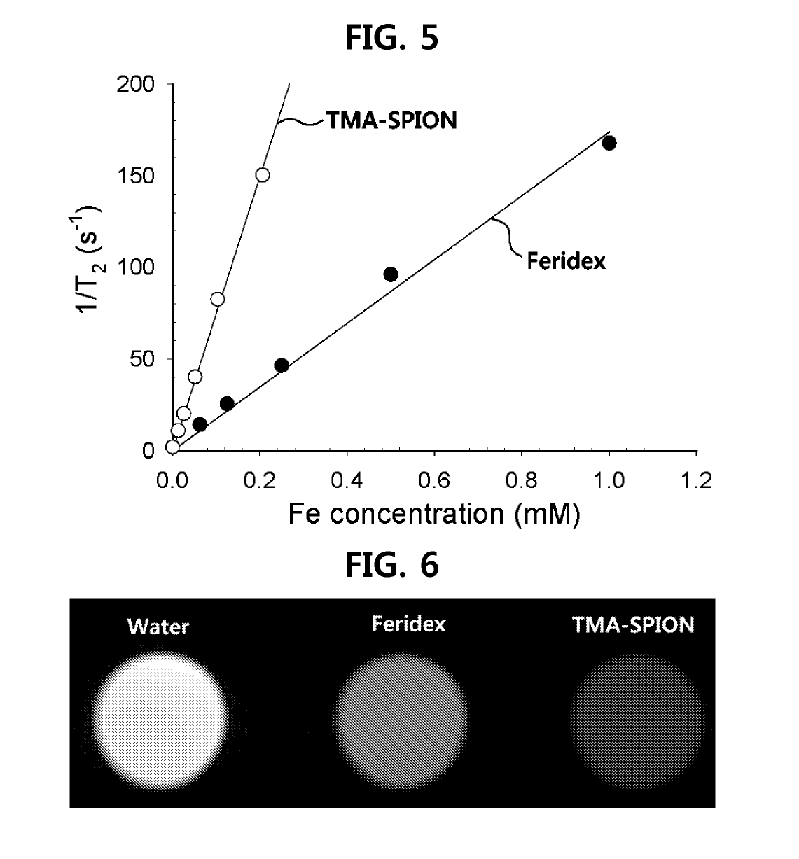

Afterwards, under the condition in which a fetal bovine serum (FBS) added to a cell culture was present, the dispersion stability of the TMA-SPIONs was tested. 0, 10, or 50 v/v% of FBSs were mixed with a phosphate buffer solution (PBS), and the TMA-SPION aqueous solution was added thereto. The resulting solution was left at room temperature, and the precipitation of the SPION was checked. Even after 22 hours, no precipitation was generated under any conditions, which indicates the dispersion stability of TMA-SPIONs (see FIG. 4).

### Analysis Example 2

### Analysis of Relaxivity

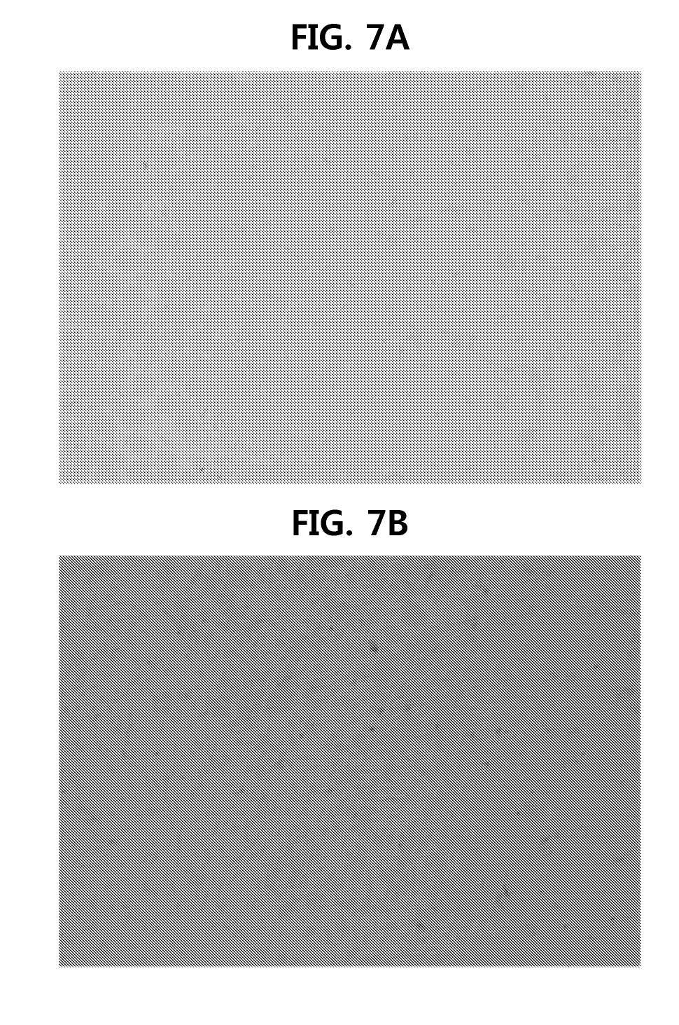

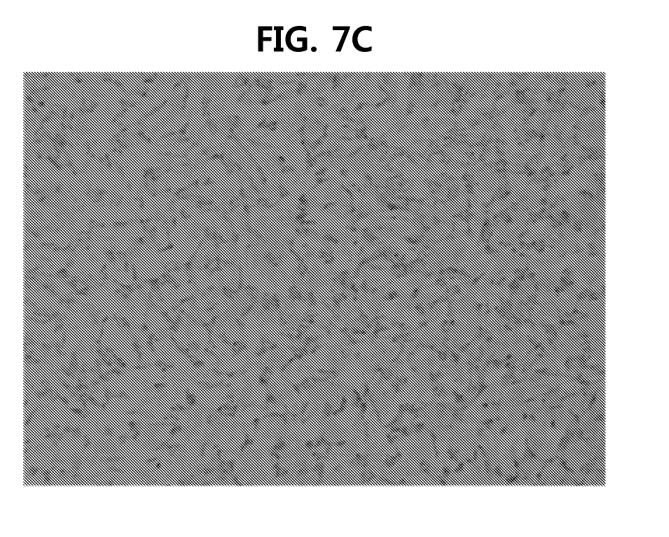

After Feridex (Advanced Magnetics, Inc.) and TMA-SPIONs were respectively dispersed in deionized distilled water, an iron concentration in each dispersed solution was measured by inductively coupled plasma-atomic emission spectroscopy (ICP-AES). As a result, Feridex was detected at 11 mg Fe/ml and the TMA-SPIONs were detected at 2.3 mg Fe/ml. After the solution was diluted with distilled water to prepare solutions with various concentrations, a 1/T2 per concentration was measured using a 7-Tesla MRI (Bruker Biospin MRI, Germany) and a relaxivity (r2) was obtained from the graph of the 1/T2 versus concentration. Compared with the r2 indicating an MR image contrasting ability of the SPION, the TMA-SPION has a 4.4 times higher value than Feridex (see FIG. 5). From the T2-weighted image (concentration of the solution=0.698 μg Fe/ml, TR=2500 msec, TE=8.5 msec) obtained by 7-Telsa MRI, it was confirmed that the TMA-SPIONs had a shorter T2 than Feridex, resulting in a negative-enhanced contrast (see FIG. 6).

### Analysis Example 3

### Analysis of Efficiency of Cell Labeling

A circular coverslip coated with polylysine (Sigma, P7890) was placed on a 12-well plate. Then, hMSCs (Lonza, PT-2501) were dispensed at a density of 1×10⁵ cells/well and incubated in a MEM alpha medium containing 10% FBS for 24 hours. Each of the Feridex and TMA-SPION dispersed aqueous solutions was diluted in 1 ml of a serum-free medium to a concentration of 0.025 mg Fe/ml, and added to the wells for a 4-hour treatment. Afterwards, the medium was exchanged with a serum-containing medium, and the cells were further incubated for 2 hours. The cells were fixed with 4% paraformaldehyde for 10 min and then washed 3 times with a phosphate buffered saline solution (PBS, 100 mM, pH 7.4, 138 mM NaCl). To analyze the labeling efficiency of the SPIONs in the cells, the cells were stained using a Prussian blue staining technique. For staining of the SPIONs in the cells, 10% acetic acid (Sigma, 32009) and 10% potassium ferrocyanide (sigma, P3289) solution were mixed into the samples, which were then rocked for 20 minutes. The stained samples were washed 3 times with PBS solution (100 mM, pH7.4, NaCl 138 mM), and to stain a nucleus and a cytoplasm, each well was treated with 1 ml of a nuclear fast red solution (Sigma, N3020) for 5 minutes. Then, the resulting cells were washed with a PBS solution, and then observed using an optical microscope of 50× magnification (Ziess, Germany) to obtain images.

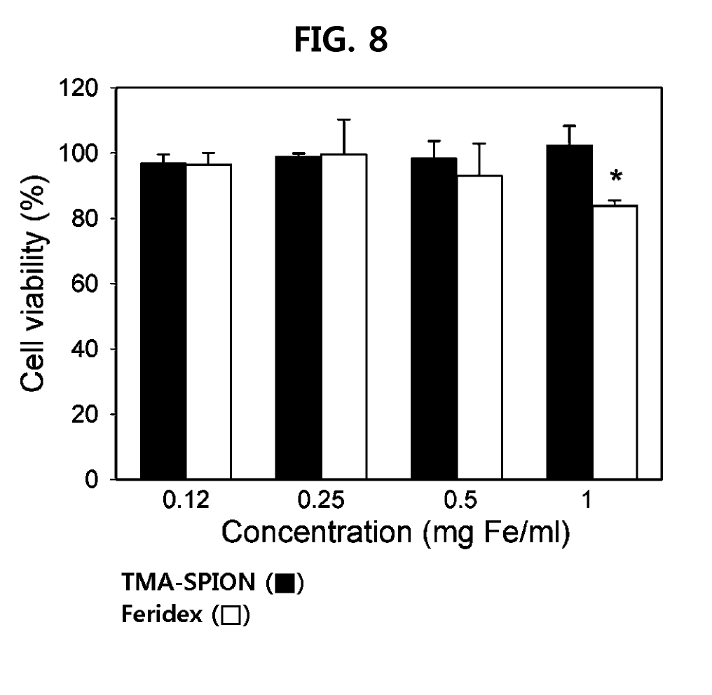

FIGS. 7A to 7C are images of hMSCs taken after the hMSCs are treated with a cell culture (control), Feridex and TMA-SPION and stained with Prussian blue (However, because FIGS. 7A to 7C are the black-and-white images, the stained areas are shown in black rather than blue). In FIG. 7A, no cells was stained in blue, but in FIG. 7B, some cells were stained in weak blue. However, in FIG. 7C, every cell was stained in dark blue, which means the TMA-SPIONs were effectively internalized into the cells. That is, it is confirmed that the conventional commercialized SPIONs require an adjuvant such as protamine sulfate that may aid intracellular penetration, but the TMA-SPIONs easily enter the cells in only 4 hours in an absence of an adjuvant. This indicates that the positive charge of the surface of TMA-SPIONs facilitates transmission to the cell.

### Analysis Example 4

### Cytotoxicity Test

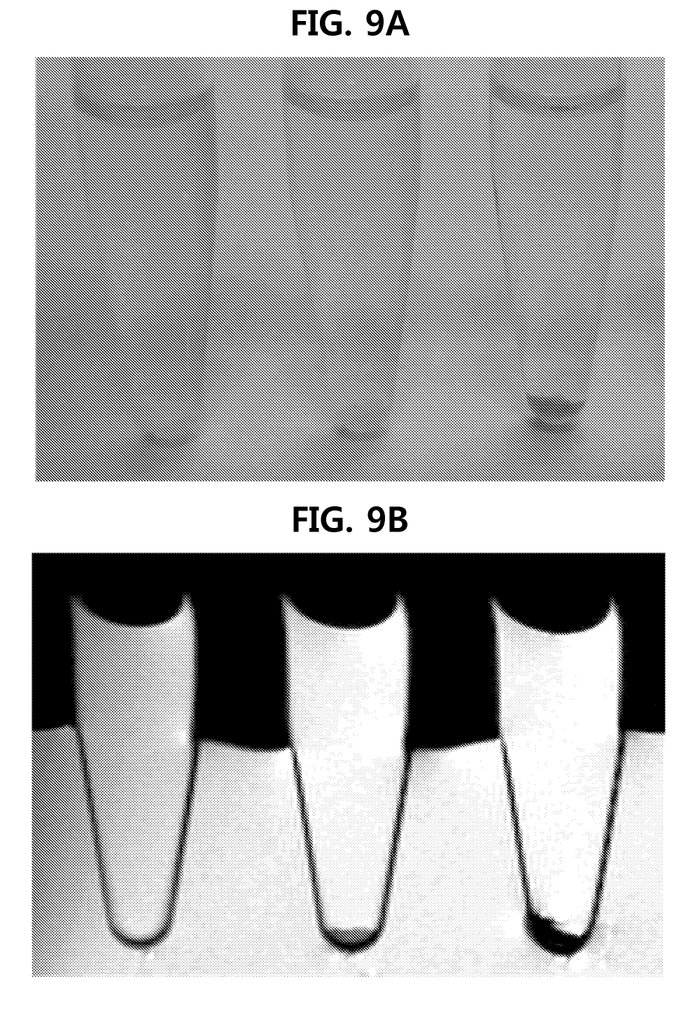

To examine cytotoxicity at various concentrations of SPIONs, each of Feridex and TMA-SPIONs was added to a serum-free cell culture medium at concentrations of 0, 0.12, 0.25, 0.5, and 1 mg Fe/ml. hMSCs were dispensed in a 96-well plate at 1×10⁴ cells/well, and incubated for 24 hours. The existing cell culture medium was removed, and a SPION-dispersed cell culture medium was added at 100 μl per well. After 4-hour incubation, cell viability was examined by CCK-8 analysis. Referring to FIG. 8, until the concentration of 0.5 mg Fe/ml, neither of Feridex and TMA-SPIONs has a significant cytotoxic effect, and Feridex shows a cell viability of approximately 80% at 1 mg Fe/ml. However, TMA-SPIONs show a cell viability of 90% or more at the same concentration (* indicates p=0.007, which is statistically significant).

### Analysis Example 5

### Comparison of T2-Weighed Images of SPION-labeled Stem Cells

To confirm a contrast effect shown from a T2-weighted MR image of SPION-labeled stem cells, 3×10⁵ of hMSCs were incubated for 24 hours in a 6-well plate, and treated with Feridex and TMA-SPIONs dispersed in a cell culture medium for 4 hours at 0.025 mg Fe/ml, respectively. A supernatant was removed, and the cells were detached from the plate by treating the cells with trypsin/EDTA (Gibco, 25200) and centrifuged for 2 minutes at 12000 rpm to obtain a cell pellet. The pellets were washed twice with PBS (100 mM, pH 7.4, NaCl 138 mM) to remove nanoparticles adsorbed on a surface of the stem cell, and a T2-weighted image was obtained using a 7-Tesla MRI system (TE=7.6 msec, TR=2000 msec, Slice Thickness=1 mm).

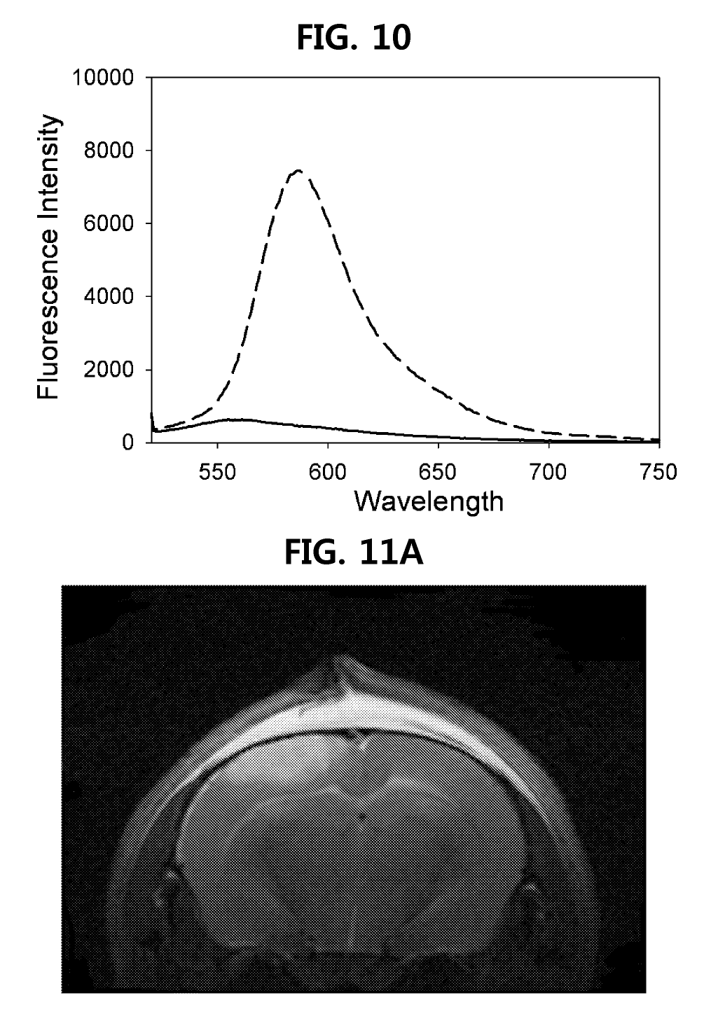

FIGS. 9A and 9B are a white-light image and an T2-weighted MR image of hMSCs labeled with SPIONs (from left, control, Feridex-treated sample, and TMA-SPION-treated sample). Referring to FIG. 9B, it is seen that TMA-SPION-labeled hMSCs show a darker T2-weighed MR image at the same concentration, compared to those labeled with Feridex.

### Preparation Example 2

### Preparation of Fluorescent Dye-Conjugated TMA-SPIONs

Fluorescent dye-bound TMA-SPIONs were prepared by the same method as described in Preparation Example 1, except the following procedure: PAA-SPIONs were treated with TMA and 5-TAMRA cadaverine (5 mg/ml, 5-carboxy tetramethyl rhodamine, AnaSpec Inc., Calif. 94555) in a ratio of 1000:1, and stirred for 30 minutes. Then, the resulting sample was treated with 0.1 M EDC (0.1 M MES buffer, pH 4.7) for 2 hours, and dialyzed in a dark room for 2 days.

### Analysis Example 6

### Fluorescence Measurement

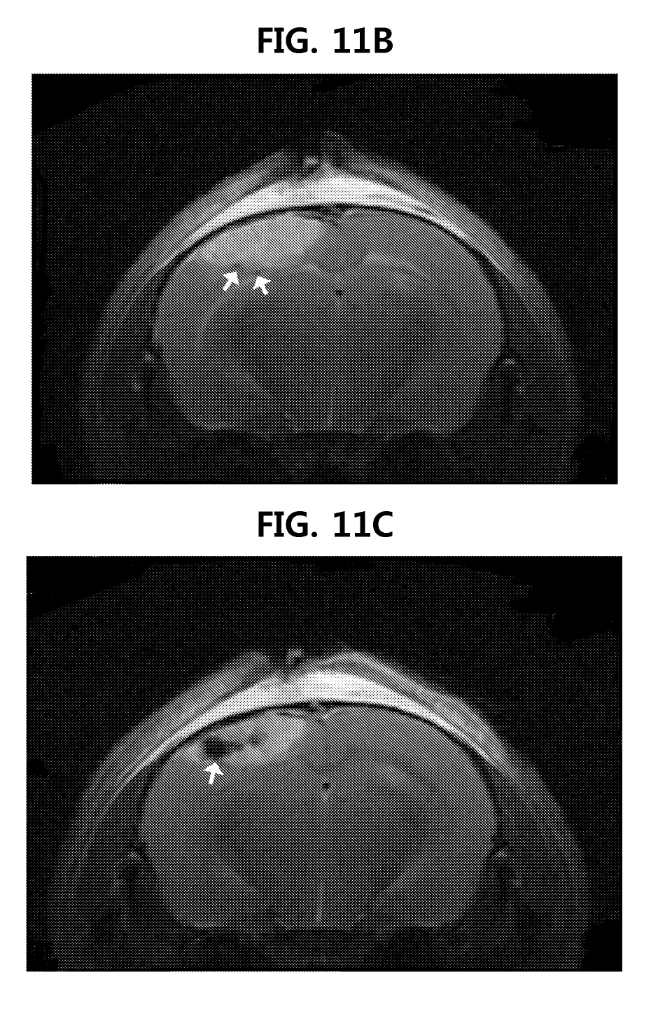

Fluorescences of the fluorescent dye-conjugated TMA-SPIONs prepared in Preparation Example 2 and fluorescent dye-free TMA-SPIONs (control) were compared. From the comparison results, it is confirmed that the fluorescent dye-conjugated TMA-SPIONs showed fluorescence intensity approximately 17 times higher than the control at 580 to 590 nm (solid line: control, dotted line: 5-TAMRA-conjugated TMA-SPION), as shown in FIG. 10. Therefore, it is confirmed that fluorescent imaging is also available as well as MRI in cells and in vivo.

### Analysis Example 7

### Evaluation of Image Tracking Efficiency of In Vivo Movement of TMA-SPION-Labeled hMSCs by MRI

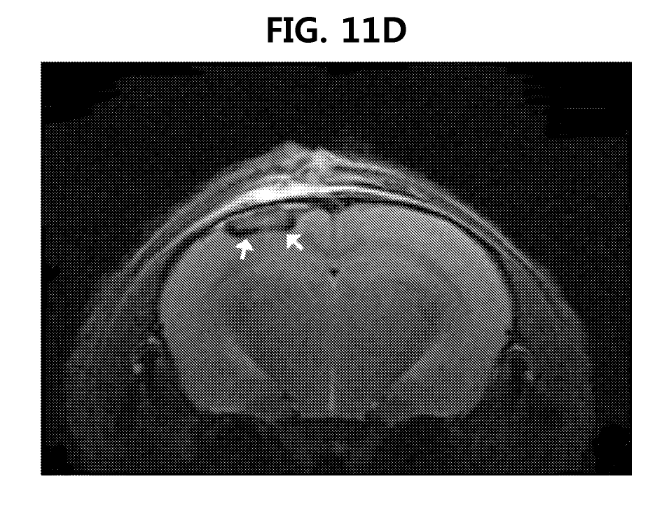

First, to prepare a brain ischemia model, 4 Balb/c-nu mice (Origin, 7˜8 weeks) were put under respiratory anesthesia, and a photosensitizer, rose bengal, was intravenously administered into a tail vein of each mouse at 5 mg/kg. A scalp of the mouse was exfoliated, and cerebral infarct was induced by irradiating 530 nm laser to a left side of the bregma at 20 mW for 10 minutes. After the laser irradiation, the scalp was sutured and stabilized for a day, and a T2-weighted MR image of the cerebral infarction region was obtained by 7-Tesla MRI (see FIG. 11A, TR=2500 msec, TE=35 msec, Slice Thickness=0.7 mm). Referring to FIG. 11A, it is seen that a cerebral model using a photosensitizer was properly prepared. Afterwards, 1×10⁵ hMSCs were treated with TMA-SPIONs at a concentration of 0.01 mg Fe/ml for 4 hours, unlabeled TMA-SPIONs were removed using a PBS, and the TMA-SPION-labeled stem cells were treated with trypsin/EDTA to isolate cells. The stem cells dispersed in a 0.05 ml medium were transferred to 0.5 ml insulin syringe (30 G, Sungsim Medical, Co., Ltd.), and intravenously administered to the tails of the mice. One, two and seven days after the injection of the TMS-SPION-labeled hMSCs, movement of the stem cells to the cerebral infarction region was examined by MRI. The MR images showed that, after the TMA-SPION-labeled stem cells were intravenously administered, some stem cells were observed in the cerebral infarction region on the first day (see FIG. 11B), and more stem cells had migrated to and accumulated in the cerebral infarction region on the second day (see FIG. 11C). It is seen that on the seventh day after the administration of the stem cells, the cerebral infarction region was recovered, and a considerable amount of stem cells were still present at the edge of the cerebral infarction region (see FIG. 11B). This shows that the MRI was effectively used to track the movement of the TMA-SPION-labeled hMSCs to the damaged cerebral site through the veins.

As described above, according to the present invention, SPIONs can be prepared in a simple and reproducible process to have hydrophilicity and a strong positive charge. The prepared positive-charged SPIONs can have high uptake into a cell and stability, and be used in various applications as an effective contrast agent through non-invasive in vivo imaging. Furthermore, it is expected that the SPIONs can be conjugated to a ligand or antibody capable of binding to a specific receptor present on a surface of a cell, thereby synthesizing various derivatives.

According to the present invention, SPIONs can be prepared in a simple and reproducible process to have hydrophilicity and a strong positive charge. The prepared positive-charged SPIONs can have high uptake into a cell and stability, and be used in various applications as an effective contrast agent through non-invasive in vivo imaging.

However, the effects of the present invention are not limited to those mentioned herein, and other effects which are not mentioned herein will be clearly understood by those of ordinary skill in the art.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A positively-charged superparamagnetic iron oxide nanoparticle (SPION), comprising:
a SPION;
a polymer layer including a polymer containing many carboxyl groups coated on a surface of the SPION; and
a cationic material coupled via an amide bond to a surface of the polymer layer.

2. The positively-charged SPION according to claim 1, wherein the positive charge has a surface zeta potential of +30 mV or more.

3. The positively-charged SPION according to claim 1, which has an average diameter of 10 to 500 nm.

4. The positively-charged SPION according to claim 1, wherein the polymer is selected from polyacrylic acid, polymethacrylic acid, polyitaconic acid and derivatives thereof.

5. The positively-charged SPION according to claim 1, wherein the cationic material is quaternary ammonium containing an amine group.

6. The positively-charged SPION according to claim 1, wherein the SPION includes maghemite (γ-Fe₂O₃) or magnetite (Fe₃O₄).

7. The positively-charged SPION according to claim 6, wherein the SPION further comprises at least one selected from manganese (Mn), cobalt (Co), nickel (Ni), zinc (Zn), and gadolinium (Gd).

8. The positively-charged SPION according to claim 1, further comprising a fluorescent dye coupled via an amide bond to a surface of the polymer layer.

9. The positively-charged SPION according to claim 8, wherein the fluorescent dye is selected from rhodamine, bodipy, Alexa Fluor, cyanine, and derivatives thereof.

10. A contrast agent comprising a positively-charged SPION according to any one of claims 1 to 9.

11. The contrast agent according to claim 10, which is used for magnetic resonance imaging (MRI), optical imaging, or MRI and optical imaging.

12. A method of preparing a positively-charged SPION, comprising:
(a) preparing a SPION whose surface is coated with a hydrophobic ligand;
(b) forming a hydrophilic polymer layer by substituting the hydrophobic ligand coated on the surface of the SPION with a polymer containing many carboxyl groups; and
(c) forming an amide bond by reaction of the carboxyl groups exposed on the surface of the hydrophilic polymer layer with the cationic material containing an amine group.

13. The method according to claim 12, wherein operation (c) further comprises forming an amide bond by reaction of the carboxyl groups exposed on the surface of the hydrophilic polymer layer with a fluorescent dye.
